(19) United States
(12) Patent Application Publication
(10) Pub. No.: US 2024/0366146 A1
(43) Pub. Date: Nov. 7, 2024

(54) SWAB-INTEGRATED MULTI-ANALYTE SENSORS FOR WOUND MONITORING

(71) Applicant: North Carolina State University, Raleigh, NC (US)

(72) Inventors: Amay Bandodkar, Raleigh, NC (US); Yong Jae Kim, Raleigh, NC (US)

(21) Appl. No.: 18/655,662

(22) Filed: May 6, 2024

Related U.S. Application Data
(60) Provisional application No. 63/464,378, filed on May 5, 2023.

(51) Int. Cl. A61B 5/00; A61B 5/103

(52) U.S. Cl. CPC A61B 5/445; A61B 5/1032

(57) ABSTRACT

Various examples are provided related to monitoring of various biomarkers in wound fluid to indicate a wound status. In one example, a wound sensing system includes a platform for examining a wound; and an array of sensors disposed on the platform. The array of sensors can include one or more fluorometric assay sensor, one or more colorimetric assay sensor, or both. Each of the fluorometric assay sensor can produce a fluorescence intensity proportional to a corresponding target concentration and each of the colorimetric sensor can produce a color change proportional to a corresponding target concentration. In another example, a method includes swiping a wound with an array of sensors of a wound sensing system; waiting for a predetermined amount of time; capturing an image of the of the array of sensors; and analyzing the image to determine one or more analyte concentration based upon fluorescence intensity, color intensity or both.

### CROSS REFERENCE TO RELATED APPLICATIONS

This application claims priority to, and the benefit of, U.S. provisional application entitled “Swab-Integrated Multi-Analyte Sensors for Wound Monitoring” having Ser. No. 63/464,378, filed May 5, 2023, which is hereby incorporated by reference in its entirety.

### BACKGROUND

Chronic, non-healing wounds are a major medical complication that can result in extended hospitalization, additional financial burden, distress, pain, amputation, and even death. Each year nearly 6.5 million individuals in the US develop chronic wounds which cost about 25 billion USD to treat. Healthcare providers presently rely on regular visual assessment of wounds to predict wound healing and chances of infection. Such rudimentary approaches severely impede clinicians from prescribing accurate treatments and is a major cause for the grim statistics involving wounds. A major issue in wound care is the lack of robust, easy-to-use, and inexpensive wound monitors that can help predict wound healing. Such knowledge can assist clinicians to identify chronic wounds early and prescribe timely and wound-specific treatments that promote rapid wound closure.

### SUMMARY

Aspects of the present disclosure are related to monitoring of various biomarkers in wound fluid to indicate a wound status. In one aspect, among others, a wound sensing system comprises a platform configured for examining a wound; and an array of sensors disposed on the platform, the array of sensors comprising one or more fluorometric assay sensor, one or more colorimetric assay sensor, or both, where each of the one or more fluorometric assay sensor is configured to produce a fluorescence intensity proportional to a corresponding target concentration and each of the one or more colorimetric sensor is configured to produce a color change proportional to a corresponding target concentration. In one or more aspects, the color change can comprise a color intensity. The platform can be a swab or a post. The platform can be a polymer membrane.

In various aspects, the wound sensing system can comprise a bandage including a slit providing access to a headspace for positioning over a wound, wherein the platform can be a strip configured to access the wound via the slit. The platform can be a flexible plastic strip. The wound sensing system can comprise one or more reference marker disposed on the platform adjacent to the array of sensors. The one or more reference marker can comprise black and white reference markers. The array of sensors can be configured to detect one or more of lactate, pH, C Reactive Protein (CRP), or hydrogen peroxide (H₂O₂). The wound sensing system can comprise a sensor configured to detect wound fluid volume.

In another aspect, a method comprises swiping a wound with an array of sensors of a wound sensing system, the array of sensors disposed on a platform and comprising one or more fluorometric assay sensor, one or more colorimetric assay sensor, or both, where each of the one or more fluorometric assay sensor is configured to produce a fluorescence intensity proportional to a corresponding target concentration and each of the one or more colorimetric sensor is configured to produce a color change proportional to a corresponding target concentration; waiting for a predetermined amount of time; capturing an image of the of the array of sensors; and analyzing the image to determine one or more analyte concentration based upon fluorescence intensity, color intensity or both. In one or more aspects, the wound can be touched for a defined period of time. The predetermined period of time can be 2 minutes or longer, or at least 15 minutes.

In various aspects, the array of sensors can be configured to detect one or more of lactate, pH, C Reactive Protein (CRP), or hydrogen peroxide (H₂O₂). The color change can comprise a color intensity. The wound sensing system can comprise one or more reference marker disposed on the platform adjacent to the array of sensors. The image can be analyzed by a device that captured the image. The array of sensors can swipe the wound through a slit in a bandage comprising headspace over the wound. The platform can be a strip configured to access the wound via the slit.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a system of swab-integrated sensors, in accordance with various embodiments of the present disclosure.

FIG. 2 illustrates an example of a fabricated swab-based system, in accordance with various embodiments of the present disclosure.

FIGS. 3A and 3B illustrate an example of a post-based system, in accordance with various embodiments of the present disclosure.

FIGS. 4A-4C illustrate an example of a sensor strip and bandage system, in accordance with various embodiments of the present disclosure.

FIG. 5 illustrates an example of a UV box for imaging of the system of FIG. 1, in accordance with various embodiments of the present disclosure.

FIGS. 6A-6G and 7 illustrate sensor testing, in accordance with various embodiments of the present disclosure.

### DETAILED DESCRIPTION

Disclosed herein are various examples of devices, systems and methods related to monitoring of various biomarkers in wound fluid to indicate a wound status. Reference will now be made in detail to the description of the embodiments as illustrated in the drawings, wherein like reference numbers indicate like parts throughout the several views.

At present there are no commercially available wound monitors. Active research is presently being pursued in developing bandage-type, wireless wound monitors. While such devices show great promise for real-time, accurate assessment of wound status, their widespread use is limited due to their reliance on complex electronics resulting in high costs. Also, their size, weight, and delicate nature make them less suitable for monitoring wounds at constricted body regions (e.g., foot, sacrum) that are most prone to developing chronic wounds. Technological restrictions also limit developing such systems to only a narrow range of analytes. The continuous presence of these sensors can also potentially interfere with the healing process causing inflammation, delayed healing, or infection.

From a user perspective, simplicity compromising on accuracy may be necessary for rapid adoption of wound monitoring diagnostics in both military and civilian domains. For example, military experts believe that the ongoing crisis in Europe and potential chances of conflict in Asia may present the US military with unprecedented battlefield scenarios characterized by delayed evacuation and high casualty density. In addition to wound monitoring, these sensors can be used for point-of-care applications in poor resource settings, especially those in which limited sample is available. Examples include environmental sensing, forensics, and food quality monitoring. Similarly, a majority of non-healing wound cases in the civilian society are among elderly, out-patient populations who do not have regular access to trained caregivers and who may have hesitation in using sophisticated sensors. Complicated, wound monitoring bandages may not be suitable for such use cases. These factors collectively limit the practical applications of bandage-integrated continuous wound monitoring systems.

The disclosed technology offers a simple, easy to use, inexpensive, yet accurate, solution that can address these limitations. Examples of devices, systems and methods are presented for simultaneous monitoring of various biomarkers in the wound fluid to acquire a comprehensive understanding of the wound status. The biomarkers can be related to, but not limited to, inflammatory, ischemic, catabolic, and infection status of wounds. The sensors can rely on quantum-dot based fluorescence and/or organic dye-based colorimetry for analyte detection. The sensors can involve enzymes; antibodies; aptamers; and/or synthetic receptors such as molecularly imprinted polymers, and/or nano/micro-particles for selective detection of an analyte. In addition to analyte-specific sensors, the device or system can also include sensors for wound fluid detection and reference markers to improve the robustness of the collected data. For example, the sensor can measure lactate, uric acid, pH, and wound fluid volume.

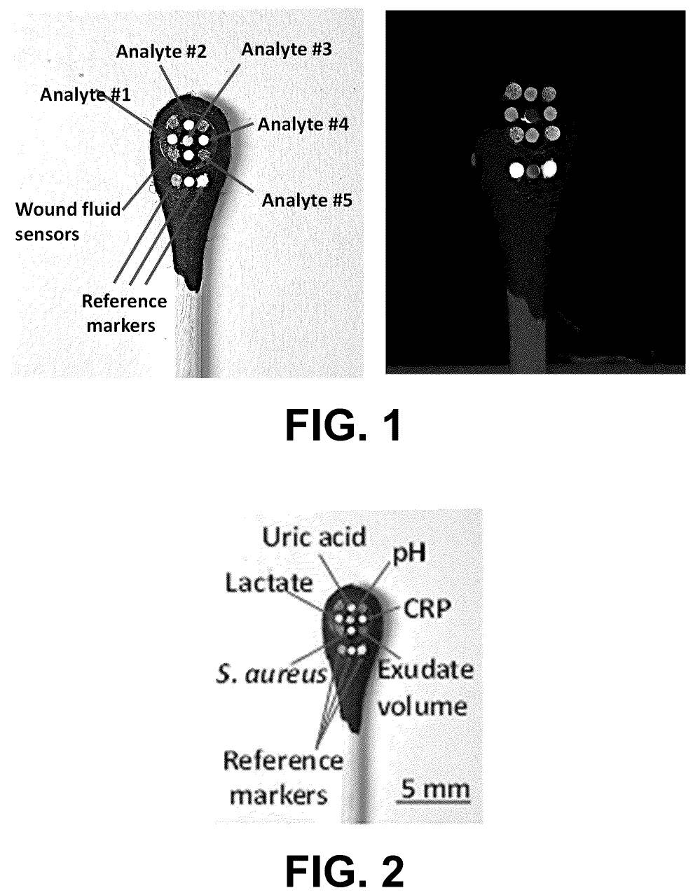

The sensors can be integrated into a swab, but other platforms or devices, such as, flexible polymer membranes, paper, bandages can also be used. Multiple chemical assays can be embedded in the swab that produce optical signal proportional to the target concentration. A first set of sensors can include assays that produce fluorescence signal while a second set can include a class of assays that produce a color change. The fluorometric assays utilize ultraviolet (UV) light (e.g., provided by a UV box or light source) for data acquisition. The colorimetric assays produce color in ambient light (without the need for special lighting). FIG. 1 includes images of a system of swab-integrated sensors in ambient light (left) and UV light (right). In the example of FIG. 1, the swab includes sensors for 5 different analytes, but the approach can be expanded to detect different numbers of analytes (more or less) as well.

Fluorometric assays: Fluorometric assays for lactate and uric acid can be fabricated by first cutting circular pads of cellulose filter paper. The pads can then be functionalized with, e.g., indium phosphide zinc sulfide (InP/ZnS) quantum-dots (QDs). Next, enzyme (lactate oxidase for measuring lactate and uricase for measuring uric acid) can be deposited by, e.g., drop casting. A layer of chitosan can be formed over the pads by drop casting followed by overnight cross linking of the chitosan polymer by, e.g., glutaraldehyde vapor. Similar approaches can be adapted for making assays for detecting C reactive protein (CRP) and S. aureus bacteria. The assay for fluid volume can be developed in a similar way except graphene QDs can be deposited on the circular pads. No enzyme immobilization step is needed for fluid volume assays.

Colorimetric assays: Hydrogen peroxide strips can be cut into 1 mm pads. The pads can then be functionalized with enzyme (e.g., lactate oxidase or uricase) and chitosan similar to steps described for the fluorometric assays. pH assay is comprised of cutting 1 mm pads of commercial pH strips.

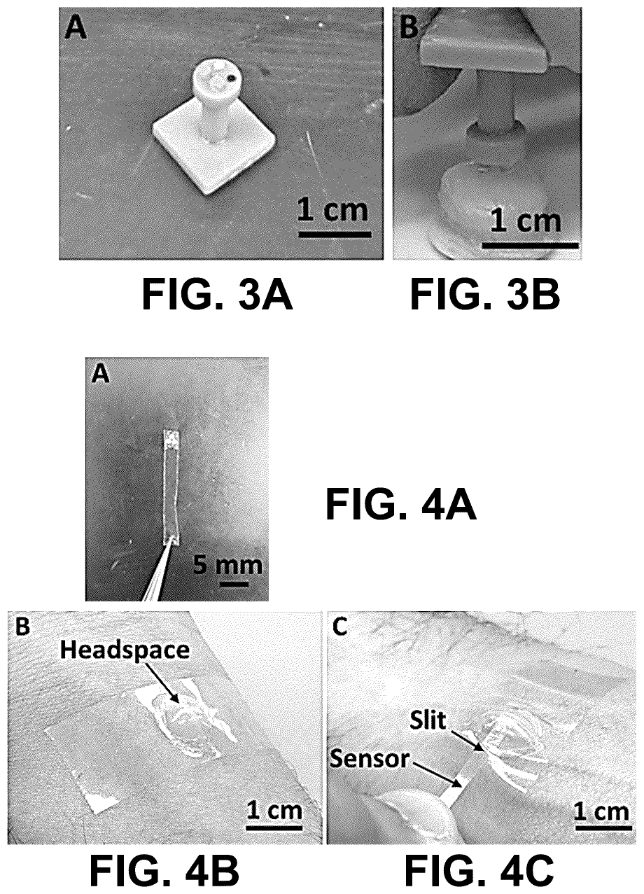

As a proof of principle, sensors for detecting wound lactate, pH, C Reactive Protein (CRP) and hydrogen peroxide (H₂O₂) were implemented. First, a swab-based platform was fabricated with the swab coated with silicone prepolymer (EcoFlex) and allowed to cure at room temperature. This ensures that the cotton fibers of the swab are coated with hydrophobic silicone layer which helps in avoiding absorption of the wound fluid by the swab. Next, a thin layer of uncured EcoFlex was applied to the swab, and the individual assays were immediately mounted on to the swab, and the EcoFlex allowed to cure. White and black color reference markers (e.g., 1 mm pads of commercially available white and black flexible plastic) were also assembled onto the swab. These reference markers help compensate for variations in lighting conditions that can affect the accuracy of the assay. FIG. 2 shows image of the fabricated swab-based system configured for sensing S. aureus, lactate, uric acid, pH, CRP, and exudate volume.

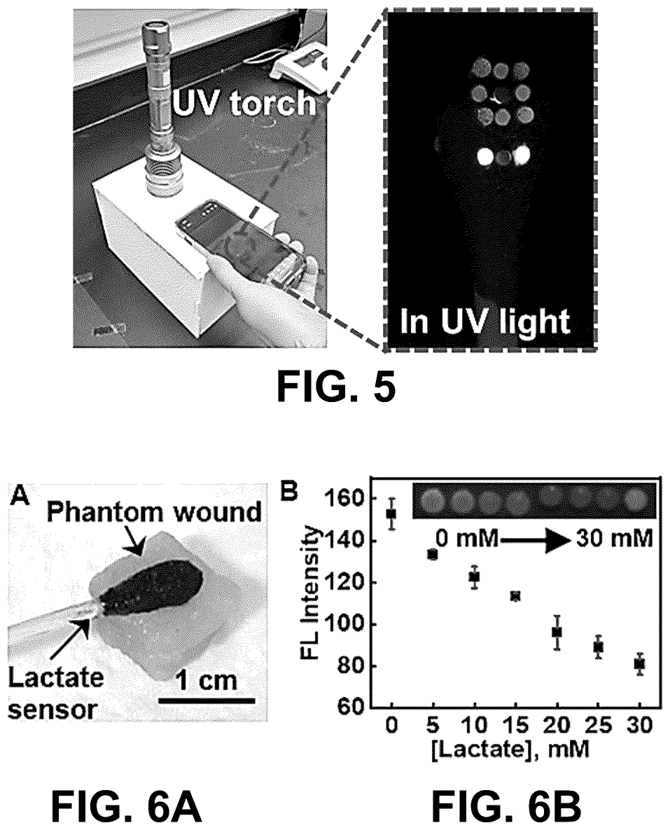

Second, customized 3D printed posts were used as the support for attaching the individual assays. FIG. 3A is an image showing assays integrated on the 3D printed post. The benefit of such posts is that it reduces the chances of shearing force that may be experienced by a swab-based system which can cause undesired removal of assay reagent. The shearing force can also damage the fragile wound bed. FIG. 3B illustrates wound fluid sampling with the functionalized 3D post using a dummy wound (on a chicken breast coated with analyte).

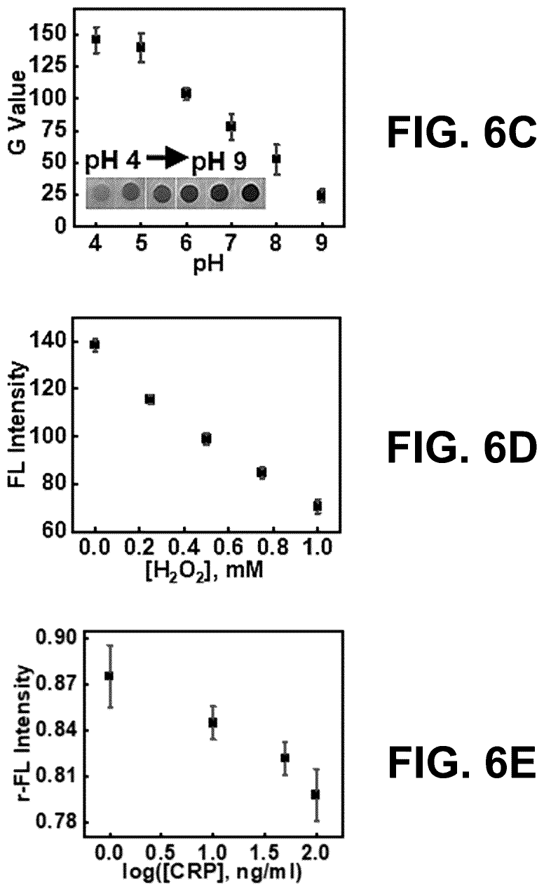

Another version of the monitoring system can include a specially designed bandage with a slit through which the sensor can be inserted into the bandage for application onto the wound. FIG. 4A-4C illustrate an example of the customized bandage with insertable flexible sensor strips. The benefit of this design is that the user does not need to remove the bandage each time they want to test the wound. FIG. 4A is an image of a flexible sensor strip. The individual assays are affixed on a thin, flexible plastic strip. The customized bandage includes a headspace with a slit on the side. The user covers the wound with the customized bandage. At the time of testing, the user slides the flexible strip-based sensor though the slit to bring the assay in contact with the wound. FIGS. 4B and 4C show the customized bandage with headspace and slit and the sensor sliding through the slit to achieve sensor-wound contact without the need for removing the bandage. As shown, the user does not need to remove the bandage every time they want to test wound.

The user can simply swipe the wound with the swab-based sensor or other sensor, wait for a for a predetermined time (e.g., a couple of minutes), and then obtain an image of the sensor using, e.g., a smartphone or tablet camera. For example, the user can gently touch the wound with the swab for 3 seconds, waits for the predetermined time, and then takes a photo of the swab. In these 3 seconds wound fluid is absorbed by the assays embedded on the swab. As the user waits for a predetermined period (e.g., 15 min for fluorescence-based assays and 2 min for colorimetric assays, the analyte molecules react with the assay reagent to produce an optical signal (e.g., fluorescence or color change). At the end of predetermined wait period, the color/fluorescence for the assay is fully developed.

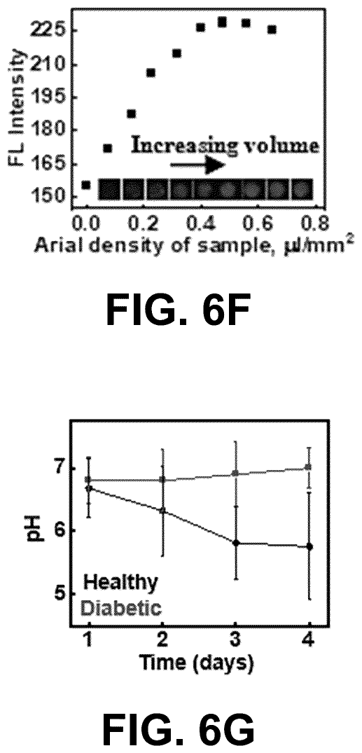

After the predetermined time, the user can take a photo of the swab using, e.g., a smartphone, tablet or other appropriate imaging device. For colorimetric assays, the image is obtained in ambient light. For fluorometric assays, the image is obtained in UV light. The UV lighting can be provided by, e.g., an UV box connected to the smartphone for data acquisition. FIG. 5 is an image illustrating an example of a custom-built UV box. In other implementations, a custom 3D printed phone case can include embedded UV LEDs to isolate the sensors from ambient light and excite the fluorescence-based sensors with UV light for signal generation.

An image analysis software (executed on, e.g., on the smartphone, tablet or a laptop or other computing device) can be used to extract color intensity of the sensors. This intensity is proportional to the analyte concentration. The results can be stored on the smartphone, tablet or computing device or can be communicated to a remotely located device where it can be accessed by concerned parties.

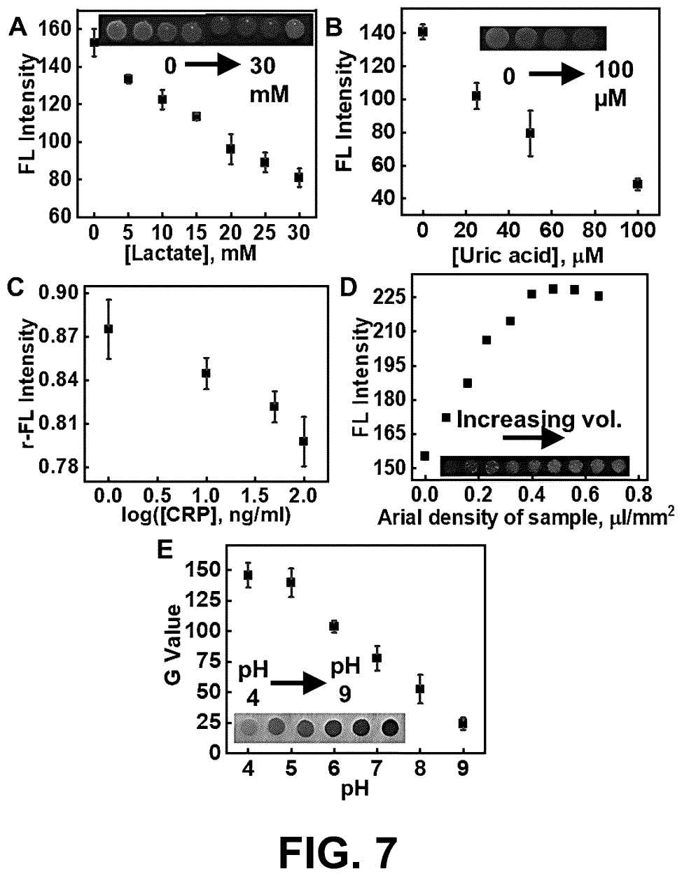

Testing of the assays for different analyte concentrations was carried out and the optical signal (fluorescence or color intensity) correlated to analyte concentration. The assay composition was optimized to acquire a linear signal response for lactate, pH, uric acid, and fluid volume in physiologically relevant concentration range. FIGS. 6A and 6B illustrate testing of lactate sensors. The image of FIG. 6A show lactate sampling on a phantom wound and FIG. 6B illustrates the fluorescence (FL) intensity as a function of lactate concentration (n=4). The inset shows images of the lactate sensors captured by a smartphone camera. FIG. 6C illustrates testing of pH sensors. The plot shows the G value (from the RGB scale) for the sensors as a function of pH (n=4). The inset shows images of the pH sensors captured by the smartphone camera. FIG. 6D illustrates testing of H₂O₂ sensors. The plot shows the FL intensity of the sensors as a function of H₂O₂ concentrations (n=4). The inset shows images of the H₂O₂ sensors captured by the smartphone camera. Additionally, a 2D heatmap of the spatial fluorescence/color intensity for individual pixels of a sensor can be obtained using image analysis software (for example, ImageJ) to estimate local density of fluid volume. This information can help in estimate would fluid volume and for accurate interpretation of analyte concentration.

FIG. 6E illustrates testing of CRP sensors. The plot shows the relative FL (r-FL) intensity of the sensors as a function of CRP concentrations (n=4). The r-FL intensity is calculated based on pre and post exposure to CRP. FIG. 6F illustrates testing of wound fluid sensors. FL intensity of the sensors is shown as a function of arial density of fluid (water) volume spread over a surface (n=4). FIG. 6G illustrates testing of animal experiments for wound pH. pH levels measured in wounds in healthy and diabetic mice are shown over a period of 4 days (n=6). FIG. 7 illustrates in vitro characterization of (A) lactate, (B) uric acid, (C) CRP, (D) exudate volume, and (E) pH assays using phantom wound (sample applied to chicken breast, n=4).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A wound sensing system, comprising:
a platform configured for examining a wound; and
an array of sensors disposed on the platform, the array of sensors comprising one or more fluorometric assay sensor, one or more colorimetric assay sensor, or both, where each of the one or more fluorometric assay sensor is configured to produce a fluorescence intensity proportional to a corresponding target concentration and each of the one or more colorimetric sensor is configured to produce a color change proportional to a corresponding target concentration.

2. The wound sensing system of claim 1, wherein the color change comprises a color intensity.

3. The wound sensing system of claim 1, wherein the platform is a swab or a post.

4. The wound sensing system of claim 1, wherein the platform is a polymer membrane.

5. The wound sensing system of claim 1, comprising a bandage including a slit providing access to a headspace for positioning over a wound, wherein the platform is a strip configured to access the wound via the slit.

6. The wound sensing system of claim 5, wherein the platform is a flexible plastic strip.

7. The wound sensing system of claim 1, comprising one or more reference marker disposed on the platform adjacent to the array of sensors.

8. The wound sensing system of claim 7, wherein the one or more reference marker comprises black and white reference markers.

9. The wound sensing system of claim 1, wherein the array of sensors is configured to detect one or more of lactate, pH, C Reactive Protein (CRP), or hydrogen peroxide (H₂O₂).

10. The wound sensing system of claim 1, comprising a sensor configured to detect wound fluid volume.

11. A method, comprising:
swiping a wound with an array of sensors of a wound sensing system, the array of sensors disposed on a platform and comprising one or more fluorometric assay sensor, one or more colorimetric assay sensor, or both, where each of the one or more fluorometric assay sensor is configured to produce a fluorescence intensity proportional to a corresponding target concentration and each of the one or more colorimetric sensor is configured to produce a color change proportional to a corresponding target concentration;
waiting for a predetermined amount of time;
capturing an image of the of the array of sensors; and
analyzing the image to determine one or more analyte concentration based upon fluorescence intensity, color intensity or both.

12. The method of claim 11, wherein the wound is touched for a defined period of time.

13. The method of claim 11, wherein the predetermined period of time is 2 minutes or longer.

14. The method of claim 13, wherein the predetermined period of time is at least 15 minutes.

15. The method of claim 11, wherein the array of sensors is configured to detect one or more of lactate, pH, C Reactive Protein (CRP), or hydrogen peroxide (H₂O₂).

16. The method of claim 11, wherein the color change comprises a color intensity.

17. The method of claim 11, wherein the wound sensing system comprises one or more reference marker disposed on the platform adjacent to the array of sensors.

18. The method of claim 11, wherein the image is analyzed by a device that captured the image.

19. The method of claim 11, wherein the array of sensors swipes the wound through a slit in a bandage comprising headspace over the wound.

20. The method of claim 19, wherein the platform is a strip configured to access the wound via the slit.
